(19) United States
(12) Patent Application Publication
(10) Pub. No.: US 2023/0321428 A1
(43) Pub. Date: Oct. 12, 2023

(54) TRANSMANDIBULAR STERILE CONDUIT

(71) Applicant: Eric Wm. Polk, Rochester, MN (US)

(72) Inventor: Eric Wm. Polk, Rochester, MN (US)

(21) Appl. No.: 18/297,540

(22) Filed: Apr. 7, 2023

Related U.S. Application Data
(60) Provisional application No. 63/362,715, filed on Apr. 8, 2022.

(51) Int. Cl. A61M 60/873; A61M 60/13; A61M 60/113; A61M 60/178; A61M 60/117; A61M 60/216

(52) U.S. Cl. CPC A61M 60/873; A61M 60/13; A61M 60/113; A61M 60/178; A61M 60/117; A61M 60/216

(57) ABSTRACT

A transmandibular sterile conduit system provides sterile access to the subcutaneous space through a conduit inserted through the mandible. The transmandibular sterile conduit system provides a sterile bacterially impervious pathway for drivelines and catheters to enter the body and connect with deeply implanted medical devices, like a Left Ventricular Assist Device, while reducing the risk of deadly infection experienced with current techniques.

### CROSS-REFERENCE TO RELATED APPLICATION(S)

This application claims priority to U.S. Provisional Application No. 63/362,715, filed on Apr. 8, 2022, and titled TRANSMANDIBULAR STERILE CONDUIT, the disclosure of which is hereby incorporated by reference in its entirety.

### BACKGROUND

Heart disease is the scourge of the modern era and remains the greatest killer of humanity worldwide. While many patients will die suddenly from acute events, many tens of thousands will wind up dying slowly from the “final common pathway” of advanced heart disease known as heart failure.

Over one-half million Americans suffer from heart failure. It is a condition in which the diseased heart can no longer pump a sufficient quantity of oxygenated blood to meet the body's needs. At first the damaged heart tries to compensate for this failure of output by growing larger. But the larger it grows, the less efficient it functions as a pump, and thus it enlarges further, setting up a vicious cycle of maladaptive responses that invariably cause an inexorable clinical deterioration in the patient that results in death.

In recent years it has become possible to extend patient's lives for several years by using complex regimens of old, and some new, drugs. But ultimately the trajectory is always downward and tens of thousands of Americans die annually from heart failure. For a lucky few the common sense solution of heart transplant will gain them many more years of life. However, due to a permanent shortage of donors only a few percentage points of the need for new hearts will ever be met by transplant.

Thus, the implanted mechanical assist device, to supplement the output of the failing heart, has been developed. These implanted mechanical pumps, such as the Left Ventricular Assist Device (known as the LVAD) help to keep the patient alive in spite of the failure of the patient's own heart. And unlike donated hearts, LVADs can be made available to all the many thousands that need them.

Unfortunately, while LVADs can and do extend life significantly, their use has been sharply limited by the fact that they do not yet extend life nearly as long as transplants of human hearts. And one significant drawback with such implanted mechanical devices is infection. In fact, as many as 30% of all LVAD implanted patients succumb to infection. One common source of these often fatal infections is the opening through which the LVAD driveline must pass between the subcutaneous space and the exterior of the patient's body.

Likewise, in those existing less invasive external ventricular assist systems using only cannulas for vascular access, infection will always enter the system. This has heretofore required a strictly short-term temporary approach for such systems, despite the fact that they are inherently far less invasive than implanted LVAD systems.

LVAD drivelines are flexible cable-like devices that deliver power and control signals to the Implanted pump. The driveline enters the body through a surgically created opening in the skin, usually on the patient's abdomen. However, human skin is colonized by millions of bacteria and with such a man-made opening to the interior of the body, the driveline itself often becomes a pathway along which harmful bacteria enter the normally sterile interior of the body. Once inside the body, bacteria have the opportunity to multiply, which may cause infections which can then travel further into the implanted device, the bloodstream, and the patient's own heart. The result is very often death. The present invention makes use of the unique properties of the human mandible and gingiva, along with the distinct ability of certain materials, (such as titanium) to fuse with bone, and the singular anatomic location of the canine tooth interspace to create a bacterially impervious appliance capable of delivering a real-world 3 mm diameter driveline or catheter to an implanted medical device, such as an LVAD or a vital interior blood vessel or organ.

In short, the present invention represents a complete transmandibular conduit system for safely delivering outside power and control to life-saving implanted medical devices while vastly reducing the risk of infection.

### SUMMARY

A transmandibular driveline or catheter conduit system includes a conduit body having a generally tubular shape with a first conduit body end and a second conduit body end. The conduit body has an outside surface including a plurality of threads and a hollow interior space passing between the first end and the second end. The conduit body is implanted in a patient's mandible and extends between the patient's oral cavity and the patient's subcutaneous space below the mandibular body. A superior conducting abutment is attached to the first conduit body end. The superior conducting abutment replaces one of the patient's teeth—preferably the left lower canine—and defines a hollow interior space between a first superior conducting abutment end and a second superior conducting abutment end. An inferior conducting abutment is attached to the second conduit body end. The inferior conducting abutment extends into the subcutaneous space and defines a hollow interior space between a first inferior conducting abutment end and a second inferior conducting abutment end. In certain embodiments, some or all of the components of the transmandibular driveline conduit system are made of titanium or any other material such as special ceramics known to be capable of osseointegration, i.e. fusing with bone into which it is implanted.

### BRIEF DESCRIPTION OF DRAWINGS

The accompanying drawings, which are incorporated in and constitute a part of this disclosure, illustrate various embodiments of the present disclosure. In the drawings:

FIG. 1 is a schematic view of one embodiment of a transmandibular sterile driveline conduit in accordance with the present disclosure showing the sterile conduit with an LVAD driveline passing from outside a patient to an implanted LVAD device;

FIG. 2 is a detail schematic view of the transmandibular sterile driveline conduit of FIG. 1 showing the positioning of the conduit after it is implanted in a human mandible;

FIG. 3 is a side schematic view of the transmandibular sterile driveline conduit of FIG. 1;

FIG. 4 is another side schematic view of the transmandibular sterile driveline conduit of FIG. 1, showing the conduit implanted in the human mandible;

FIG. 5 is a schematic view of one step of the surgical method of implanting the transmandibular sterile driveline conduit of FIG. 1, showing the position of an incision to provide access to the mandible;

FIG. 6 is a schematic view of one step of the surgical method of implanting the transmandibular sterile driveline conduit of FIG. 1, showing the extraction of a patient's canine tooth;

FIG. 7 is a schematic view of one step of the surgical method of implanting the transmandibular sterile driveline conduit of FIG. 1, showing the positioning of a boring tool as it passes through the patient's mandible;

FIG. 8 is another schematic view of the step of FIG. 7, showing the step of boring a hole through the patient's mandible at the gumline;

FIG. 9 is a schematic view of one step of the surgical method of implanting the transmandibular sterile driveline conduit of FIG. 1, showing the implantation of one embodiment of a healing abutment at an inferior end of the sterile driveline conduit in accordance with the principles of this disclosure;

FIG. 10 is a schematic view of one step of the surgical method of implanting the transmandibular sterile driveline conduit of FIG. 1, showing the implantation of a healing abutment at a superior end of the sterile driveline conduit;

FIG. 11 is a schematic view of one step of the surgical method of implanting the transmandibular sterile driveline conduit of FIG. 1, showing the implantation of one embodiment of an inferior conducting abutment in accordance with the principles of this disclosure;

FIG. 12 is a schematic view of one step of the surgical method of implanting the transmandibular sterile driveline conduit of FIG. 1, showing the implantation of one embodiment of a superior conducting abutment in accordance with the principles of this disclosure;

FIG. 13 is a schematic view of one step of the surgical method of implanting the transmandibular sterile driveline conduit of FIG. 1, showing the positioning of a driveline through the fully implanted conduit;

FIG. 14 is another schematic view of a driveline passing through the superior conducting abutment;

FIG. 14A illustrates another example in which a sterile transmandibular conduit system may also be employed in tandem to accommodate a complete externally mounted minimally invasive LVAD system;

FIG. 15 is a perspective view of one embodiment of a healing abutment for a transmandibular sterile driveline conduit in accordance with the principles of the present disclosure;

FIG. 16 is a side view of the healing abutment of FIG. 15;

FIG. 17 is a bottom view of the healing abutment of FIG. 15;

FIG. 18 is a perspective view of one embodiment of a superior conducting abutment for a transmandibular sterile conduit in accordance with the principles of the present disclosure;

FIG. 19 is a front view of the superior conducting abutment of FIG. 18;

FIG. 20 is a side view of the superior conducting abutment of FIG. 18;

FIG. 21 is another side view of the superior conducting abutment of FIG. 18;

FIG. 22 is a perspective view of one embodiment of an inferior conducting abutment for a transmandibular sterile driveline conduit in accordance with the principles of the present disclosure;

FIG. 23 is a front view of the inferior conducting abutment of FIG. 22;

FIG. 24 is a side view of the inferior conducting abutment of FIG. 22;

FIG. 25 is another side view of the inferior conducting abutment of FIG. 22;

FIG. 26 is a top view of one embodiment of a transmandibular sterile driveline conduit in accordance with the principles of this disclosure;

FIG. 27 is a side view of the transmandibular sterile driveline conduit of FIG. 26, showing the conduit passing through the patient's mandible;

FIG. 28 is another side view of the transmandibular sterile driveline conduit of FIG. 26, showing one embodiment of a cutting abutment attached to a first end of the conduit and one embodiment of a handle attached to a second end of the conduit in accordance with the principles of this disclosure;

FIG. 29 is a side view of one embodiment of a cutting abutment in accordance with the principles of this disclosure;

FIG. 30 is a section view of the cutting abutment of FIG. 29, taken generally along the line 30-30 in FIG. 29;

FIG. 31 is a top view of the cutting abutment of FIG. 29;

FIG. 32 is a side section view of one embodiment of a superior driveline connector in accordance with the principles of this disclosure, showing the connector in a partially disassembled configuration;

FIG. 33 is another side section view of the superior driveline connector of FIG. 31 in a fully assembled configuration; and

FIG. 34 is a schematic perspective view of the superior driveline connector of FIG. 31 in the fully assembled configuration.

### DETAILED DESCRIPTION

The present disclosure relates generally to medical devices. In particular, the present disclosure relates to a system for providing sterile vascular access that may be used for providing electrical power and control to implanted medical devices (such as LVAD or artificial heart pump) while reducing the likelihood of infection.

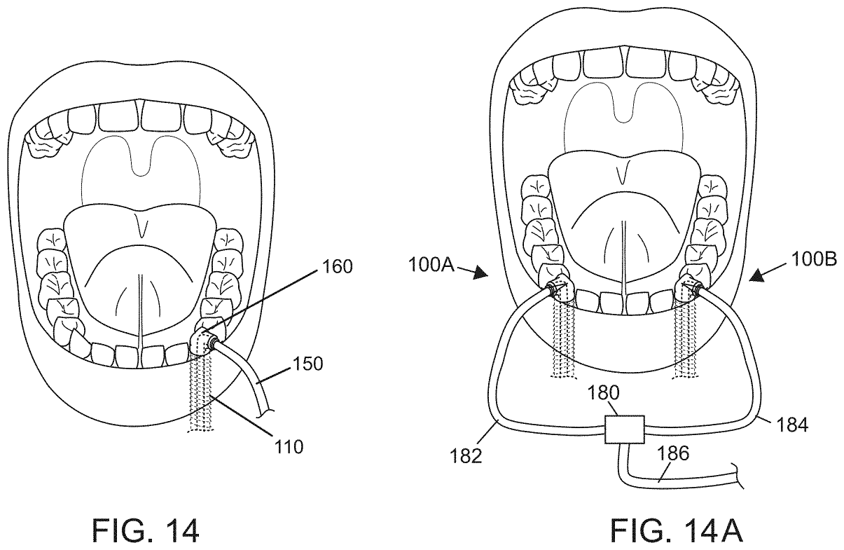

Similarly, the conduit system described herein may be used as a sterile conduit to give catheters sterile access to interior body vessels as, for example, in those patients on dialysis in whom AV fistulas can not be effectively maintained. The sterile transmandibular conduit system may also be employed in tandem, one on the right—for pump input—and one on the left of the mandible—for pump output. This arrangement will create an extracorporeal, externally mounted, fully portable LVAD system that can be instituted with minimally invasive techniques, thereby making the benefits of ventricular assistance available to a vastly broader group of patients, especially those too frail to withstand the major surgery of a fully implantable pump system. An example of such an arrangement is illustrated and described in further detail with reference to FIG. 14A.

A transmandibular conduit provides a passage through the mandible to the interior of a body. By combining the unique properties of the metal titanium with the unique anatomy and tissues of the human mandible, it becomes possible to create a new device which allows the introduction of a catheter or driveline into the interior of the body with a significant reduction of the deadly 30% incidence of—sometimes fatal—infections seen heretofore. It is expected that most patients would consider the provision of an oral—rather than abdominal—flesh-colored 3 mm diameter driveline to not be an insurmountable inconvenience in exchange for potential years of added life free of deadly device infection. In certain embodiments, an ‘anti-drooling’ device can be offered to those who might benefit from such a modality during the hours of sleep. The end result is saved lives, a vastly increased survival time and, above all, a crucially important step toward the goal of making the LVAD an everyday modality in the fight against end-stage heart failure.

The human mandible 200 (or jawbone) is characterized by some unique tissues and a biological environment not seen anywhere else in the human body. It is the only area of the body in which the body allows an eruption of hard tissues from interior bone to come exposed to an outside surface filled with millions of bacteria, and still prevent deadly infections from entering the site(s) of the eruptions from the contaminated outside environment. Those eruptions from inside the body are known as teeth and the unique soft tissue which prevents bacteria from traveling along the teeth into the sterile interior of the body is known as gingiva (or gums).

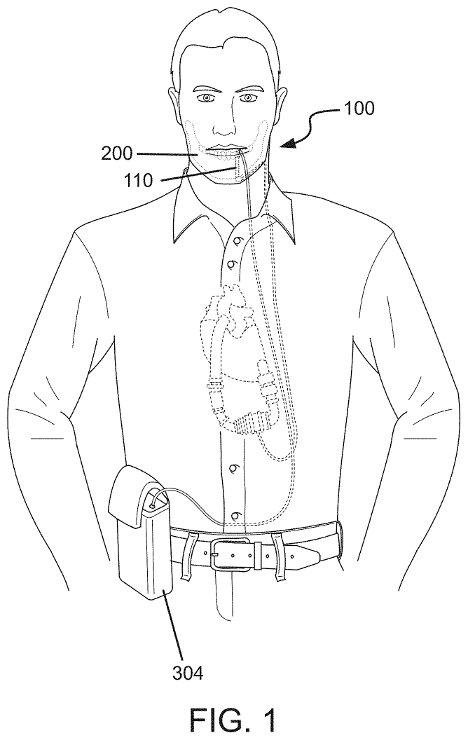

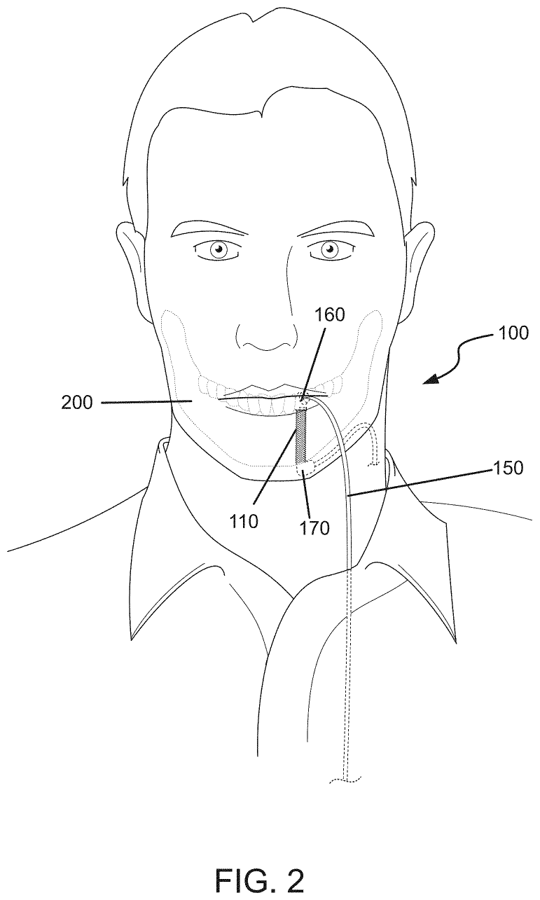

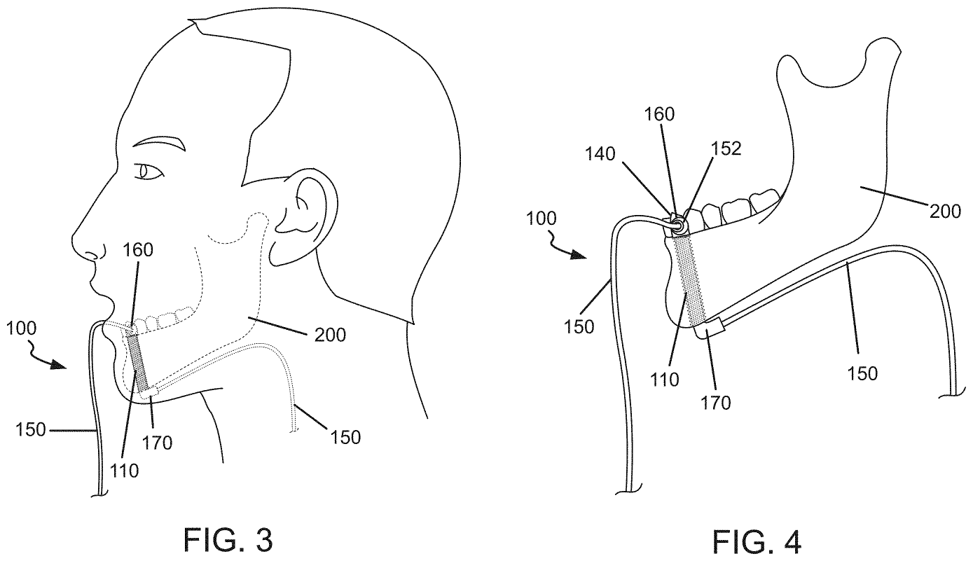

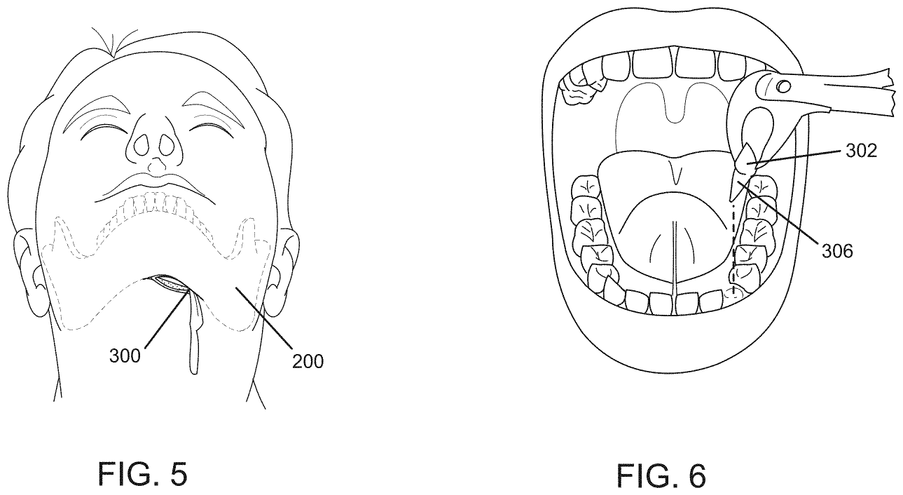

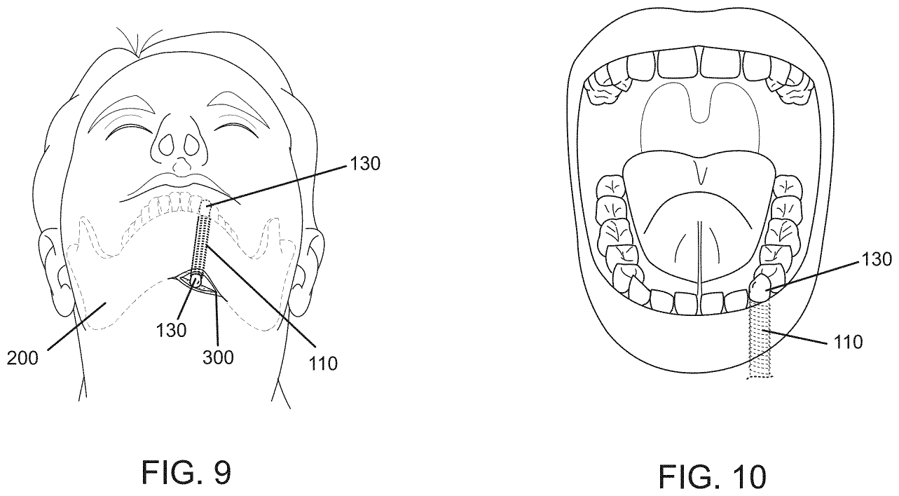

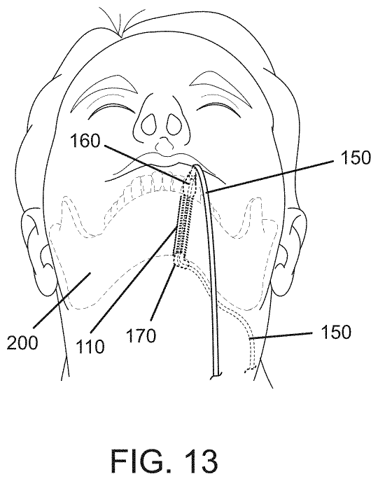

FIGS. 1-14 illustrate one embodiment of a method for implanting a transmandibular sterile conduit system 100 in accordance with the principles of this disclosure. The described configuration has been designed to accommodate a conventional 3 mm LVAD driveline or catheter for dialysis, (or, in tandem with a contralateral conduit implant, a fully portable extracorporeal circulatory assist system). In such case, a conduit body 110 is designed to be implanted into the mandible at the root track of the left mandibular canine tooth 302. The unique geometry of that location permits convenient access to an LVAD and also allows the implanted conduit system to avoid disruption of the neurovascular bundle in the alveolar canal while at the same time avoiding contact with the mental nerve.

The present disclosure relates a system of components which—when implanted strategically into a prepared site in the mandible 200—will provide the patient with a stable, secure, sterile and impervious conduit for an outside-the-body vascular catheter or driveline to supply power and control to an LVAD or any other suitable medical device. Titanium is a metal having unique properties when implanted into the human body. Specifically, when implanted into bone, titanium undergoes a process of fusing with the host tissue known as osseointegration. Osseointegration results in a strong, stable, impervious and permanent fusion of the titanium device with the host bone.

By making use of the unique properties of titanium—combined with the characteristics of gingival tissue and mandibular anatomy—it becomes possible to implant a transmandibular sterile conduit system 100 into the mandible 200 from above—through a predrilled track extending along the space previously occupied by the root of a tooth. The surgical method for implanting the transmandibular sterile conduit system 100 is described in detail below.

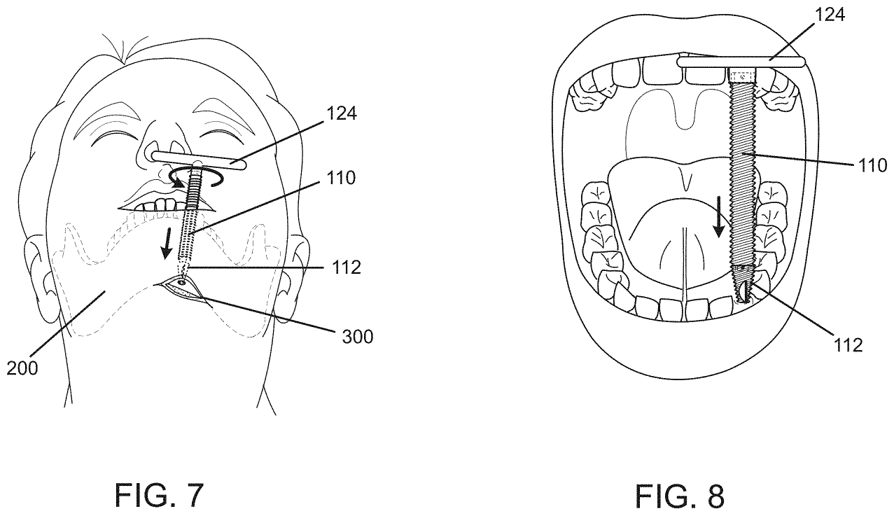

As shown in FIG. 6, after a patient has been prepped and anesthetized for surgery, the first step of implanting the transmandibular sterile conduit system 100 is the extraction of the patient's left mandibular canine tooth 302 (when present) to provide a suitable location to implant the transmandibular sterile conduit system. As shown in FIGS. 7 and 8, a track is drilled along the course of the left mandibular canine tooth 302 to provide a path for the transmandibular sterile conduit system 100 to pass through the mandible 200.

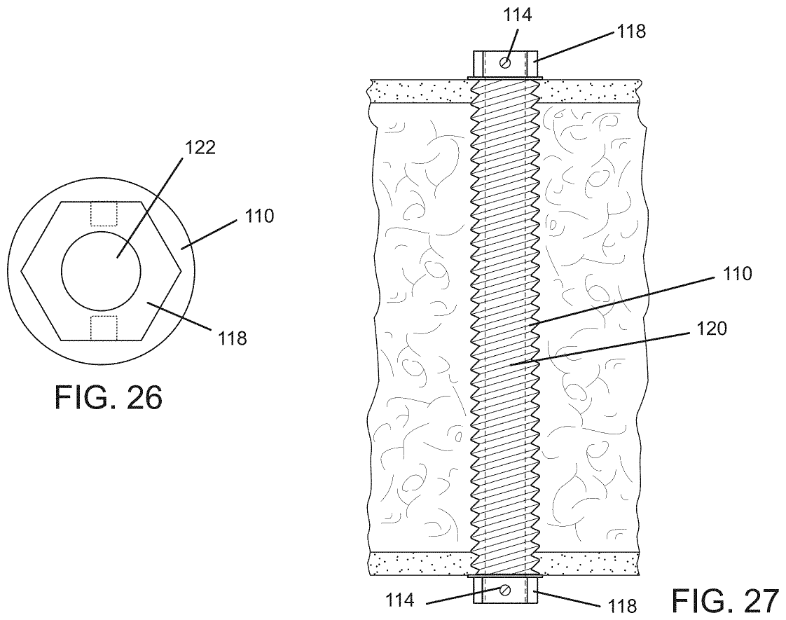

Transmandibular sterile conduit system 100 includes an approximately 40 mm long driveline conduit body 110 that is designed to emerge from the cortical bone 202 of the inferior aspect 204 of the mandible 200 in the sterile subcutaneous—below the skin—space 206. In certain embodiments, driveline conduit body 110 may be longer or shorter to accommodate mandibles of varying sizes. Driveline conduit is shown in detail in FIGS. 26 and 27. As shown, driveline conduit body 110 includes a threaded portion that passes through the mandible. Driveline conduit body 110 includes a hollow interior space 120 that passes through the conduit and provides a path through which an external driveline 150 can pass.

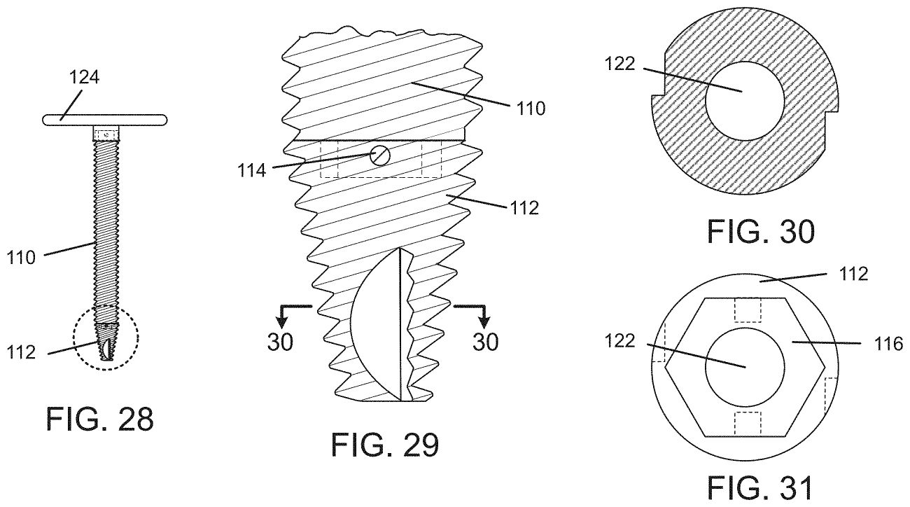

Next, firm implantation of the transmandibular sterile conduit system 100 is achieved by removably attaching a cutting abutment 112 to a leading—inferior—end of the driveline conduit body 110. Cutting abutment 112 is shown in greater detail in FIGS. 28-30 and is utilized at the time of surgical implantation following preparation of the implant site along the course of the left canine root 306. In certain embodiments, cutting abutment 112 is attached and fixed with set screws 114 to the inferior aspect of the driveline conduit body 110. In certain embodiments, cutting abutment 112 further includes a female hexagonal (hex) shaped recess 116 that selectively engages a male hexagonal (hex) shaped end 118 of conduit body 110. The hexagonal shapes of the recess 116 and end 118 prevent rotation of the cutting abutment 112 in relation to the driveline conduit body 110. In certain embodiments, hex shaped recess 116 and hex shaped end 118 may have alternative corresponding shapes without departing from the principles of this disclosure. In certain embodiments, cutting abutment 112 is hollow, with a cutting abutment channel 122 that aligns with hollow interior space 120. Hex shaped recess 116 mates with the hex shaped end 118 of conduit body 110 at the inferior end of the conduit. In certain alternative embodiments, conduit body 110 may include threaded ends to allow cutting abutment 112 to be threaded onto the conduit.

Turning back to FIGS. 7-8, the surgeon positions the cutting abutment 112 and conduit body 110 assembly at the top of the aforementioned implantation track through the mandible 200 and rotates the cutting abutment 112 and conduit body 110 assembly by turning a handle 124 that is temporarily attached to the end of the conduit body opposite the cutting abutment, causing it to progressively enter the track. The engagement of the hexagonal shapes of the end 118 of the conduit body 110 and the recess 116 of the cutting abutment 112 prevent rotation of the cutting abutment in relation to the conduit body as it is driven into the mandible 200. Once the cutting abutment 112 and conduit body 110 assembly passes through the mandible 200, it emerges into the subcutaneous space 206 below the mandible. Access to the emerging cutting abutment 112 is obtained through a small submandibular incision 300. Once the implanted cutting abutment 112 and conduit body 110 assembly emerges from the inferior mandible 200 the now exposed cutting abutment is removed by loosening set screws 114 and separating the cutting abutment 112 from conduit body 110.

The superior end of the conduit body 110 extends above the cortex of the superior aspect of the mandible 200 where it terminates in a configuration that allows the attachment of a healing abutment 130 which extends approximately 6 to 7 mm above the gingiva (or gums) just short of the hight of adjacent dentition.

Similarly, at the time of implant, a suitably shaped healing abutment 130 is placed upon the inferior end of the conduit body 110. In certain embodiments, healing abutments 130 are coated with a silicone elastomer to promote formation of a smooth subcutaneous pocket. Access to the site of the inferior healing abutment 130 is obtained via the above referenced small submandibular incision which is then closed following placement of the abutment.

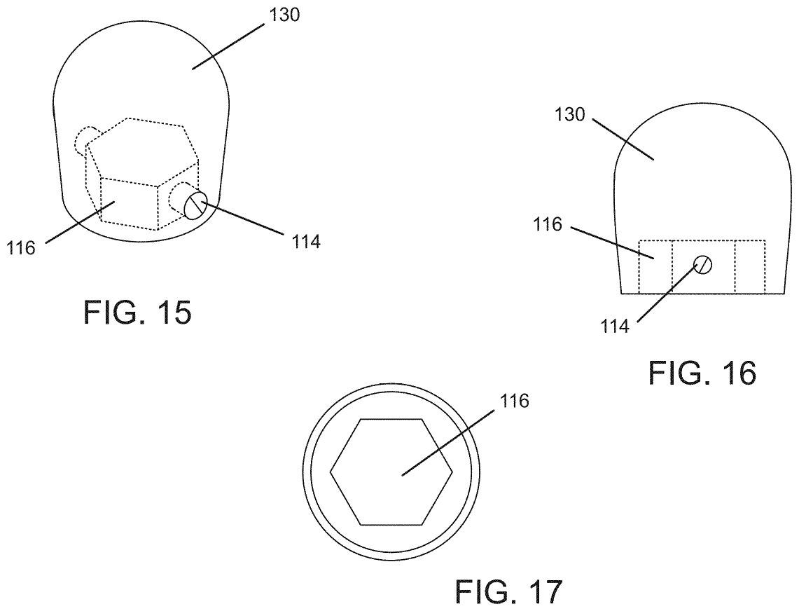

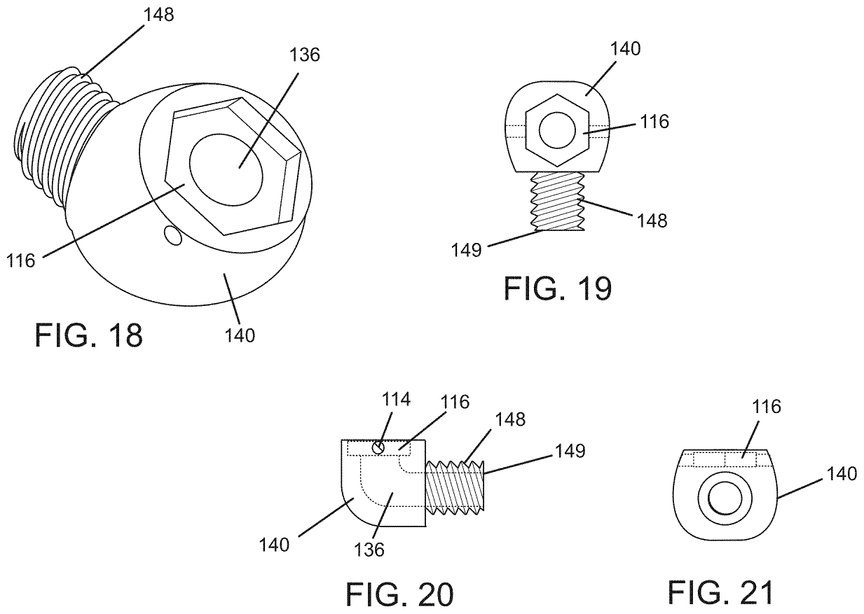

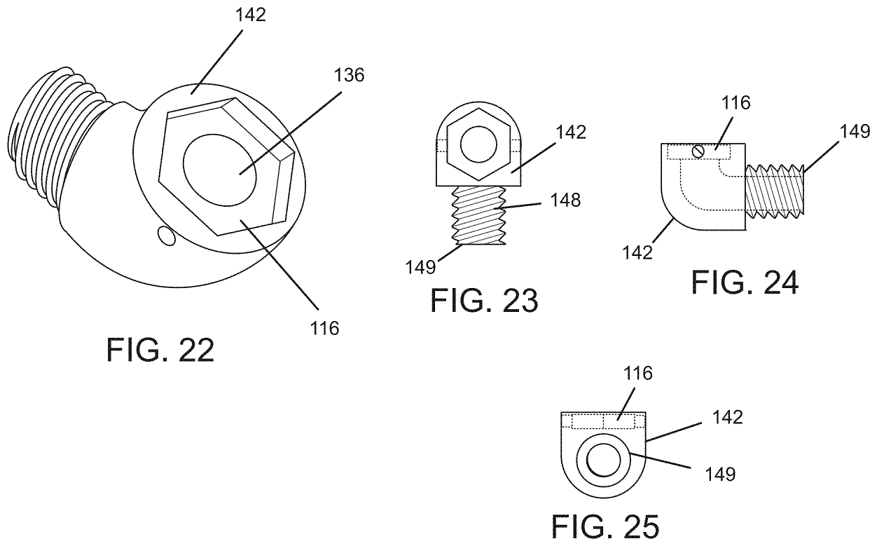

As shown in FIGS. 15-17, healing abutment 130 is designed to be attached to either the superior or inferior end of implanted conduit body 110 and enable full healing and stabilization of the conduit prior to placement of the driveline itself and the LVAD it is controlling. In certain embodiments, healing abutment 130 may be tooth shaped and colored to match a patient's existing dentition. Each healing abutment 130 has a hex shaped recess 116 secured, in certain embodiments, by two titanium M 0.6 set screws 114 of 0.7 mm length to the hex shaped ends 118 of the conduit body 110. Similar to the cutting abutment 112, the engagement of the hexagonal shapes of the end 118 of the conduit body 110 and the hex shaped recess 116 of the healing abutment 130 prevent rotation of the healing abutment in relation to the conduit body. The healing abutment 130, as well as the permanent superior conducting abutment 140 (described below) should be provided in a range of physiological heights so that any occlusal or masticatory function is avoided. In certain embodiments, set screws 114 may be longer or shorter and have larger or smaller diameters. Put plainly, healing and conducting abutments 130, 140 should extend from the gum line a distance that avoids contact with opposing teeth.

The purpose of the healing abutments 130 is to seal off and protect the hollow interior space 120 of the conduit body 110 for approximately six weeks while the body heals and both the cortical bone 202 and trabecular bone 203 of the mandible 200 fuse to the implanted titanium conduit forming an impervious, immovable, solid device for driveline or catheter placement.

During the approximately six week healing period, the gingival tissues in the mouth will heal and conform to the tooth-like geometry of the healing abutment 130. While it is clearly understood in the world of dentistry that such healed gums surrounding, for example, a prosthetic tooth implant does not exhibit the identical histology or dental ligamentous attachment of a normal biological tooth, the body does in fact manage to almost always create an effective bacteriologically secure seal to the implant.

Thus, following the appropriate healing period the patient has a solid, stable and bacteriologically impervious conduit that can now be used to introduce a sterile driveline into the body at the time of LVAD placement. On the day of LVAD placement, while the patient is under general anesthesia, the healed implanted conduit body 110 is prepared for permanent service.

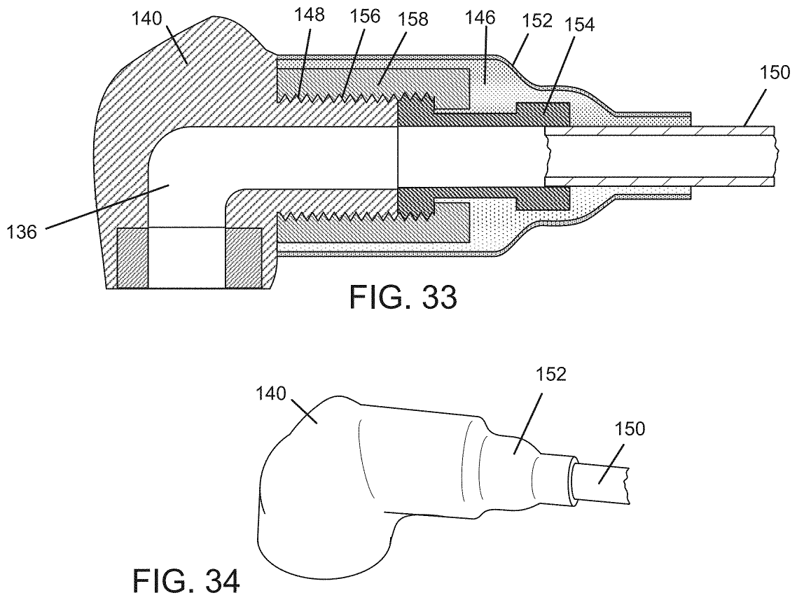

At this point both healing abutments 130 are removed and a permanent superior conducting abutment 140 is threaded upon an external driveline 150. Prior to feeding an external driveline 150 through the conduit body 110, it will be provided in sterile condition and will be threaded in the operating room with, first, a conducting abutment sheath 152 (see FIG. 33) followed by a coaxial sealing connector 154. In certain embodiments, the conducting abutment sheath 152 is made of 0.25 mm thick titanium, although alternative materials may be used without departing from the scope of this disclosure.

FIGS. 31-33 show embodiments of the superior and inferior conducting abutments 140, 142. In certain embodiments, the conducting abutments 140, 142 include a bore 136 that defines a path for driveline 150 (or, for that matter, a vascular access catheter for dialysis, etc.). The bore 136 has a diameter of approximately 3 mm and passes through threaded projection 148 which emerges from flat machined table 149 which surrounds the base of the machined threaded projection. The superior conducting abutment 140 is designed to point in a perpendicular direction in relation to the mandibular body so that the driveline or catheter exits the oral cavity to the left of center (but not so far off center that perleche becomes an issue).

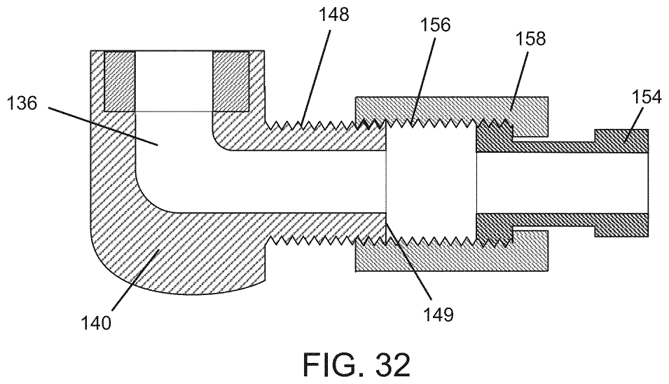

FIG. 32 shows coaxial sealing connector 154 poised to be tightened and fixed upon a machined threaded projection 148 of the permanent superior conducting abutment 140. This connection is achieved by rotation until the coaxial sealing connector 154 mates with the flat machined table 149 at the base of the threaded projection 148. Next, the surgeon attaches and tightens two titanium set screws 114 in the proximal segment of the superior driveline connector. The next step is to rotate the distal segment of the coaxial sealing connector 154 until it forms a tight seal with the distal machined surface of the machined threaded projection of the superior conducting abutment 140. Note that the junction between the coaxial sealing connector 154 and superior conducting abutment 140 is now encapsulated within the internal threads 156 of superior driveline connector 158.

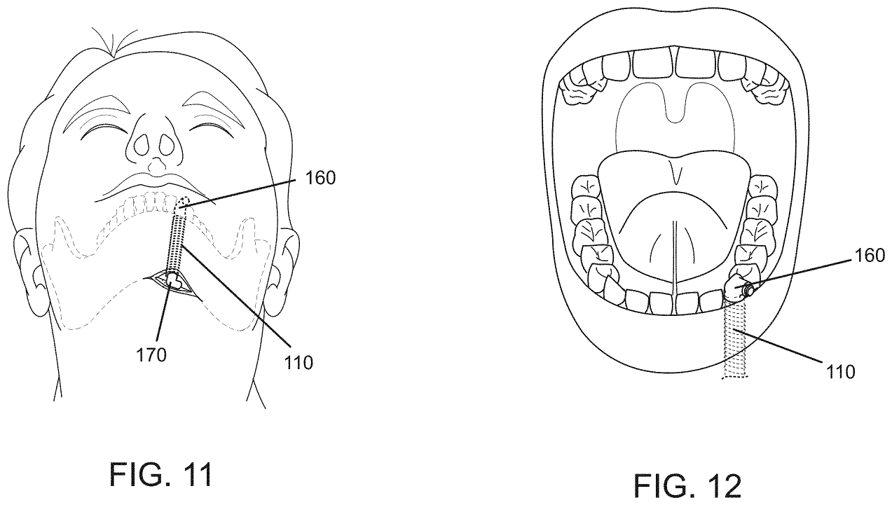

Next, the driveline 150 is threaded through the implanted conduit body 110 where it emerges in the sterile subcutaneous space below the mandible 200. As shown in FIG. 11, access to the site is again obtained through a small incision through which the driveline 150 can temporarily emerge. At that point the driveline 150 is allowed to protrude from the mandible 200 the desired distance and is then threaded with a permanent inferior conducting abutment 142, followed by an inferior coaxial sealing connector 144 and then a conducting abutment sheath 152.

FIG. 4 shows one embodiment of a complete inferior conducting abutment assembly 170. The completed inferior conducting assembly 170 is similar in principle to the superior conducting assembly 160 described in detail above. However, there is no need for the inferior conducting abutment 142 to mimic dental architecture and color matching and abutment hight considerations are not of critical concern in this location. The external surfaces of the entire inferior conducting abutment 142 and its associated conducting abutment sheath 152 may be supplied pre-coated with a black or neutral silicone coat. In certain embodiments, a finishing band of elastomer is applied to conducting abutment sheath 152 extending approximately 5 mm on either side of the distal and proximal terminations of the conducting abutment sheath. Following placement of the permanent abutment on the conduit body 110, inferior conducting abutment 142 should be pointing proximally along the axis of the mandibular body.

The entire sterile transmandibular driveline conduit system 100 has now been assembled and is ready for permanent fixation. The superior conducting abutment 140 includes a hex shaped recess 116 that is inserted upon the hex shaped end 118 on the superior aspect of the conduit body 110 and secured with set screws 114. As described above, the engagement of the hexagonal shapes of the end 118 of the conduit body 110 and the hex shaped recess 116 prevents rotation of the superior conducting abutment 140 in relation to the conduit body. In certain embodiments, the set screws are made of titanium. The coaxial sealing connector 154 is then slid down the driveline 150 and rotated upon the threaded projection 148 of the superior conducting abutment 140 until it tightly and firmly covers the threads and mates with the flat machined table 149 of the permanent superior conducting abutment 140. In certain embodiments, an internal rotating lock section 155 of the coaxial sealing connector 154 is then rotated tightly forward until it presses against and seals off the distal end of the threaded projection of the superior conducting abutment 140 (which is, itself, now sealed completely within the main body of the coaxial sealing connector 154).

Next, the surgical field is well irrigated and all detritus cleared from the permanent superior conducting abutment assembly 160. While protecting the oral environment with a rubber dam, fast-setting potting compound 146 is painted onto the exterior of the now mounted and tightened coaxial sealing connector 154. In certain embodiments, following tightening and fixation of the superior driveline connector 158, a generous quantity of the same potting compound 146 is introduced by injection into the interior of the conducting abutment sheath 152 which is then attached to the exposed coaxial sealing connector 154 and slid down upon the superior driveline connector 158. A generous amount of potting compound 146 should be utilized to ensure that all internal surfaces of the sheath 152 and all external surfaces of the coaxial sealing connector 154 and superior driveline connector 158 are well coated with potting compound. Excess potting compound is removed and scraped off all adjacent parts and materials prior to full cure. A rubber dam may optionally be utilized during assembly to avoid contamination of the oral environment with excess compound. The completed superior conducting abutment assembly 160 may then be brushed with a fast-cure skin-toned silicone elastomer coating which shall extend over and 5 mm beyond the distal and proximal ends of the sheath 152. In certain embodiments, the skin-toned coating of silicone elastomer may extend from 2 mm beyond the proximal end of the sheath 152 to approximately 25 mm beyond the junction of the distal sheath and the driveline 150 (which itself should be skin-toned). It is anticipated that drivelines, too, be provided in a range of skin tones to promote patient acceptance of the several inches of driveline that would always be visible after implantation of the transmandibular sterile conduit system 100. The superior part of the transmandibular sterile conduit system 100 is now assembled, fixed, tightened and sealed and is now fully impervious to bacterial invasion compared to a conventional driveline or vascular catheter entering the body.

Attention is now directed to the inferior aspect of transmandibular sterile conduit system 100. Inferior conducting abutment 142 is slid down the driveline 150 and seated upon the inferior hex shaped end 118 of the conduit body 110 where it is fixed with two set screws 114. Again, the engagement of the hex shaped end 118 of the conduit body 110 and the hex shaped recess 116 prevents rotation of the inferior conducting abutment 142 in relation to the conduit body. Care is taken to assure the inferior conducting abutment 142 follows the same direction, proximally, as the mandibular body so that the driveline 150 is well positioned to be directed centrally toward the LVAD or other implanted appliance. In a similar fashion to the above described superior conducting abutment 140, the coaxial sealing connector 154 is fixed and tightened to the inferior conducting abutment 142 by rotation onto the threaded projection 148 of the abutment until it mates tightly with the flat machined table 149 of the abutment. The internal rotating lock section 155 is then similarly tightened and sealed firmly against the distal threaded projection of the inferior conducting abutment 142, which is, itself, now sealed completely within the main body of the coaxial sealing connector 154.

The application of fast setting potting compound to the interior of the inferior conducting abutment sheath is similar to the above described procedure. Note that there is no need for skin-toned silicone elastomer since the inferior conducting abutment 142 exists hidden in its own sterile subcutaneous space and may thus be supplied in advance with the exterior of the conducting abutment sheath 152 pre-coated with silicone. Nonetheless a generous sealing coat of elastomer should be applied to both the proximal and distal ends of the conducting abutment sheath 152 where it meets the exterior of the inferior coaxial sealing connector 144 and the driveline 150 respectively.

The entire sterile transmandibular conduit system 100 has now been implanted, fixed, sealed and locked in place. It now only remains for the surgeon to employ a conventional subcutaneous tunneling tool to create a path to thread the driveline or catheter 150 in a conventional manner to its termination at the LVAD or other implanted appliance. While individual applications will vary, a well designed removable connector can be provided before termination at the LVAD and/or, at the other end, before entering an external power or control module 304. By such well known modalities it becomes possible to repair, replace or service parts of a system without having to disrupt or explant the entire system.

In the case of other configurations or morbidities requiring more than one sterile access to the interior of the body (such as permanent sterile vascular access for dialysis) the contralateral mandibular canine root track may be utilized for a second access line. Similarly, an adjacent tooth site may be utilized provided proper care has been taken preoperatively to verify the fact that the proposed adjacent conduit does not impinge upon the alveolar canal or mental nerve.

Given the fact that individual patients vary in their anatomic dimensions it is clear that implanted conduits 110, while averaging 40 mm in length, sans abutments, must be provided in a suitable range of lengths so that the surgeon may select the exact length suitable to the geometry of the individual patient mandible. Similarly, both of the superior healing and conducting abutments 130, 140 are designed to have no occlusal function and should be provided in a physiologic range of hights so that the surgeon may select an abutment which stands approximately 1 mm short of surrounding dentition. These considerations are irrelevant for the inferior abutments 130, 142, which may be provided in one standard configuration.

Patients should be instructed on the details of maintaining good oral hygiene and the wisdom of avoiding hard crunchy foods. Patients should understand that carrying a flexible permanent 3 mm skin-toned driveline protruding from the side of their mouth is expected to be no more obtrusive than the pipe always locked between Grandpa's lips or the cigar always present clenched in the teeth of Uncle Al. Of course, the equivalence is not exact and the patient must be given the ultimate choice: a hidden conventional driveline (or vascular access for dialysis) or a transmandibular sterile driveline conduit system 100 which reduces the expected incidence of infection significantly. In either case the patient must always be made to understand that a leading cause of death with implanted devices that use conventional external lines is infection. Armed with such information the patient is then in a position to make an informed choice.

The size and shape of driveline conduit body 110 may be adjusted or modified for different clinical settings, as, for example, an emergency placement in which it is thought best to implant the entire complete transmandibular sterile driveline conduit system 100 in one procedure at the time of LVAD placement, rather than utilizing the more conservative staged process described herein. In such a situation thread, pitch, chamfer and angle architecture can be modified to obtain, for example, maximum purchase and the highest degree of postoperative mechanical stability prior to osseointegration. In either case the surgeon must be provided a selection of conduit bodies 110 in physiologic lengths so that he/she can match the implanted conduit bodies 110 to the dimensions of the patient's mandible at the left and/or right canine root.

FIG. 14A illustrates another example in which a sterile transmandibular conduit system may also be employed in tandem to accommodate a complete externally mounted minimally invasive LVAD system. The conduit system described herein may be used as a sterile conduit to give catheters sterile access to interior body vessels as, for example, in those patients on dialysis in whom AV fistulas can not be effectively maintained. The sterile transmandibular conduit system may also be employed in tandem, one on the right (100A)—for pump 180 input—and one on the left (100B) of the mandible—for pump output. This arrangement forms an extracorporeal, externally mounted, fully portable LVAD system that can be instituted with minimally invasive techniques, thereby making the benefits of ventricular assistance available to a vastly broader group of patients, especially those too frail to withstand the major surgery of a fully implantable pump system.

More specifically, the example in FIG. 14A includes a first transmandibular conduit system 100A (e.g., on the patient's right side) coupled to a inflow line 182, which provides circulatory inflow to an external pump 180. The external pump 180 is schematically represented by a rectangular box. In some embodiments, the external pump 180 is an extracorporeal circulatory assist pump. The pump 180 may be supported (such as by a necklace or other support such as a strap or band), for example, so that it is suspended below the collar. An external drive line 186 (extending downward from the pump 180 in FIG. 14A) can be used to supply power to the pump, and can be connected to a power source (not shown) or control module (e.g., 304, shown in FIG. 1). A second transmandibular conduit 100B (e.g., on the patient's left side) is coupled to an output of the pump 180 and provides a pump outflow line 184, such as to assist a failing heart, increasing blood flow and blood pressure throughout the body.

Although the embodiments herein described are what are perceived to be the most practical and preferred embodiments, this disclosure is not intended to be limited to the specific embodiments set forth above. Rather, modifications may be made by one of skill in the art of this disclosure without departing from the spirit or intent of the disclosure.

What is claimed is:

1. A transmandibular driveline conduit system comprising:
a conduit body having a generally tubular shape with a first conduit body end and a second conduit body end, the conduit body having an outside surface including a plurality of threads and a hollow interior space passing between the first end and the second end;
the conduit body implanted in a patient's mandible, the body extending between the patient's oral cavity and the patient's subcutaneous space;
a superior conducting abutment attached to the first conduit body end, wherein the superior conducting abutment replaces one of the patient's teeth and defines a hollow interior space between a first superior conducting abutment end and a second superior conducting abutment end; and
an inferior conducting abutment attached to the second conduit body end, wherein the inferior conducting abutment extends into the subcutaneous space and defines a hollow interior space between a first inferior conducting abutment end and a second inferior conducting abutment end.

2. The transmandibular driveline conduit system of claim 1, wherein the first and second ends of the conduit body define a hexagonal shape.

3. The transmandibular driveline conduit system of claim 2, wherein the superior and inferior conducting abutments define hexagonal shaped recesses that correspond to the hexagonal shape of the first and second ends of the conduit body.

4. The transmandibular driveline conduit of claim 1, further comprising a LVAD driveline passing through the hollow spaces of the superior conducting abutment, the conduit body, and the inferior conducting abutment.

5. The transmandibular driveline conduit of claim 1 a superior driveline connector attached to the superior conducting abutment.

6. The transmandibular driveline conduit of claim 1, wherein the conduit body has a length of approximately 40 mm.

7. The transmandibular driveline conduit of claim 1, wherein the inferior conducting abutment includes a driveline connector.

8. The transmandibular driveline conduit of claim 1, wherein a conducting abutment sheath covers at least a portion of the superior conducting abutment.

9. The transmandibular driveline conduit of claim 1, wherein a conducting abutment sheath covers at least a portion of the inferior conducting abutment.

10. The transmandibular driveline conduit of claim 1, further comprising a coaxial sealing connector selectively connected to a driveline connector, each of the coaxial sealing connector and the driveline connector including a channel through which a driveline or catheter may pass.

11. A method for implanting a sterile transmandibular conduit comprising the steps of:
boring a hole through a patient's mandible at the patient's gumline, the hole providing a pathway from the patient's oral cavity to the patient's subcutaneous space below the mandible;
inserting a conduit body through the hole, the conduit body having a generally tubular shape with a first conduit body end and a second conduit body end, the conduit body having an outside surface including a plurality of threads and a hollow interior space passing between the first end and the second end, the hollow interior space defining a path from the patient's oral cavity to the patient's subcutaneous space below the mandible;
temporarily attaching a healing abutment to the first conduit body end and the second conduit body end;
allowing the conduit body to undergo osseointegration into the mandible and gum tissue;
removing the healing abutment from the first conduit body end and the second conduit body end;
threading a driveline through the hollow interior space of the conduit body; and
attaching a conducting abutment to the first and second conduit body ends.

12. The method of claim 11, further including the step of sealing the conducting abutment to the conduit body ends using an epoxy potting compound.

13. The method of claim 11, further including the step of selecting a conduit body having a length that corresponds to the size of the patient's mandible.

14. The method of claim 11, further including the step of pre-threading the driveline through the conducting abutment prior to attaching the conducting abutment to the first and second conduit body ends.

15. The method of claim 11, further including the step of attaching a conducting abutment sheath to the conducting abutment that covers at least part of the conducting abutment.

16. The method of claim 11, further including the step of coating the inferior abutment sheath with silicone.

17. The method of claim 11, further including the step of attaching the superior and inferior conducting abutments to the first and second conduit body ends by at least one set screw.

18. The method of claim 11, wherein the first and second conduit body ends define a hexagonal shape.

19. The method of claim 18, wherein each of the superior and inferior conducting abutments define a hexagonal recess that corresponds to the hexagonal shaped ends of the first and second conduit body ends.

20. The method of claim 11, wherein the conduit body has a length of approximately 40 mm.
